(19) United States
(12) Patent Application Publication
(10) Pub. No.: US 2007/0213591 A1
(43) Pub. Date: Sep. 13, 2007

(54) Disposable Cap for Endoscope

(75) Inventors: Amram AIZENFELD, Kibbutz Ramot Menashe (IL); Golan SALMAN, Tirat Hacarmel (IL); Omer SHEZIFI, Haifa (IL); Reuven ZAMIR, Yokneam Illit (IL)

Correspondence Address: BRUCE E. LILLING;LILLING & LILLING PLLC, P.O. BOX 560, GOLDEN BRIDGE, NY, 10526, US

(73) Assignees: STRYKER GI LTD., Haifa (IL); Sightline Technologies Ltd.

(21) Appl. No.: 11/470,103

(22) Filed: Sep. 5, 2006

Related U.S. Application Data
(60) Provisional application No. 60/714,480, filed on Sep. 6, 2005.

(51) Int. Cl. A61B 1/06; G02B 26/02

(52) U.S. Cl. 600/175; 600/176; 600/177; 359/229

(57) ABSTRACT

A cap, which is detachably connectable to an optical head of an endoscope, has a tubular housing, which is manufactured by injection molding from translucent plastic material. The housing includes a rear open end and a frontal end closed by a frontal face and an antiglare ring for preventing a viewer of the optical head from viewing parasitic reflections. The antiglare ring is embedded within the frontal face of the cap and said optical head can view an object of interest through a portion of the frontal face which is confined by the antiglare ring. The antiglare ring is provided with at least one discontinuity region through which the plastic material can flow during injection molding of the cap.

### BACKGROUND OF THE INVENTION

1. Field of the Invention

The present invention relates to the field of endoscopy and specifically to a cap attachable to an optical head of an endoscope, which can be used in medical applications e.g. as a colonoscope. The colonoscope is used in colonoscopic procedures, during which a flexible tube provided with the optical head is inserted into the rectum and colon to enable visualization and examination of the colon interior for abnormalities. It should be borne in mind, however, that the present invention is not limited strictly to caps employed in colonoscopes. The present invention covers any other endoscopic apparatuses used for the purpose of examination, operation, diagnostic, therapy etc. Among such endoscopic apparatuses one can mention endoscopes for examination of the esophagus, stomach and duodenum, cystoscopes for examinating the bladder, angioscopes, bronchoscopes, laparoscopes, arthroscopes, sigmoidoscopes etc. Furthermore the present invention covers not only medical, but also industrial applications, and is applicable in industrial endoscopes, or so called boroscopes.

2. Summary of the Prior Art

There are various known endoscopes employing optical heads for visualization of the interior of the body cavity or lumen. Essential parts of such optical head are the imaging system and the illumination system. The imaging system might comprise an objective at the distal end of the endoscope or a fiber bundle and an eyepiece at the proximal end of the endoscope to observe the interior of the lumen with the eye.

In the modern endoscopic devices, the imaging system comprises an imaging optic and an imager chip, e.g. in the form of a CCD-chip or CMOS, which transforms the reflected light signals into electric signals, passing to the proximal end via electric lines and visually presented, as a real image, on an image reproduction unit outside the endoscope.

In the further disclosure the objective, the fiber bundle with an eyepiece or the CCD-chip or CMOS will be referred-to as an image viewer.

The illumination system serves for transmitting light to the distal end of the endoscope for illuminating the location to be observed. Such illumination system might comprise external light sources, e.g. xenon or halogen light sources, or internal light sources, e.g. light emitting diodes (LED's) located within the endsocope.

The use of CCD sensors and LED's in an optical head of an endoscope is relatively new issue, nevertheless one can mention many patents describing endoscopes provided with such optical devices.

An example of an endoscope with LED illumination can be found in Nakashima (U.S. Pat. No. 6,533,722). In this endoscope the LED's are located within the endoscope shaft and at a distal end thereof.

Another example of an endoscope provided with a CCD-chip and LED's is disclosed in Irion (U.S. Pat. No. 6,730,019). In this endoscope the imaging system is also located within the shaft, while the LED's are arranged at the proximal end of the endoscope outside from the shaft.

The present invention concerns an optical head, in which, preferably, but not exclusively, a CCD camera with a projective lens is employed as an image viewer and LED's provide a light source.

The modem endoscopes might employ also a sheath or sleeve, which covers an endoscope insertion tube to prevent its contamination during the endoscopic procedure.

An example of such an endoscope can be found in Eizenfeld (WO 2004/016299; International patent application PCT/IL2003/000661), which disclosure is incorporated herein by reference.

In this application is described a colonoscopic apparatus provided with an insertion tube, covered by a protective sleeve. A cap seals the distal end of the insertion tube and the distal end of the sleeve is connected to the cap. An opening is provided at the distal end of the cap to enable viewing the interior of the body channel during the endoscopic procedure. A window made of transparent material closes the opening.

Unfortunately use of the transparent window is associated with the so-called ghost image problem or glare problem. Unnecessary or so-called parasitic reflections entering the CCD camera through projective lens cause this problem. The reason for unnecessary reflections can be, for example, reflection of light from an inside surface of the window or reflections within the window itself. Accordingly the projective lens percepts not only the light, which has been reflected from the relevant objects representing a true image, but also parasitic reflections, which deteriorate the image rendering it less sharp and less coherent.

This problem is known and there exist various attempts to solve it.

So, for example, in Silverstein (U.S. Pat. No. 5,193,525) is disclosed an antiglare tip for a sheath of an endoscope. The tip is provided with a dedicated interfitting region ensuring that the image viewer percepts only light reflected from objects external to the endoscope.

The disadvantage of this solution is associated with the necessity to arrange within the interfitting region dedicated mating projections and recesses on the tip and on the distal end of the endoscope. To achieve the desired antiglare effect one should ensure that these projection and recesses have very accurate dimensions, which means that during their manufacturing very narrow tolerances have to be met. Obviously, this renders the manufacturing process complicated and expensive.

There exists also an endoscopic apparatus developed by Sightline Technologies Ltd. (now known as Stryker GI Ltd) that is marketed under the trademark ColonoSight™. This apparatus comprises an insertion tube covered by a disposable, inflatable sleeve, and the distal end is secured on a plastic cap, which accommodates therein an optical head. The frontal face of the cap is provided with a transparent window to enable view for the image viewer, i.e. the projective lens of a CCD-chip. There is provided also an antiglare ring, situated around the projective lens.

The cap is manufactured from a plastic material, e.g. ABS (Acrylonitrile Butadiene Styrene), by injection molding within a die. Before injection the antiglare ring is placed within the die and, when the cap is being injected, the antiglare ring remains within the die. When the ready cap is removed from the die, the ring becomes embedded within the frontal face of the cap. Since the ring is shaped as a closed circle, the plastic material, which is being injected in the die, cannot approach the area encircled by the ring. This area remains empty and is sealed by a transparent window during the next step of the manufacturing process. This step includes careful placement of the window within a depression made in the antiglare ring so as to locate the window concentrically therewith. The further step is filling of the annular groove between the window and the ring by a glue to secure the window in place. This manufacturing process is dictated by the present construction of the cap, namely by the fact that the window is separate from the cap. One should appreciate that this manufacturing process is complicated, inconvenient and costly since it requires several steps.

Furthermore, since the window is separate from the cap, a distance should be provided between the window and the lens, otherwise there is a danger that the lens will protrude too much towards the windows and might even have displaced it from the depression. Since the light rays pass through this space and refract, their exit points are located close to the window edge. This might result in catching irrelevant objects outside the window. Therefore, the window should be located within the depression very carefully and accurately, otherwise reflections from the irrelevant objects might enter in the field of view. Obviously this requirement is associated with the necessity to comply with strict tolerances on the dimensions of the elements of the cap, which renders the manufacturing process even more complicated.

Thus, despite known attempts to devise a cap for an endoscope optical head, nevertheless, there still exists a need for a new and improved cap, which can be manufactured easily, economically and conveniently and which would still have the required antiglare properties.

In particular it would be desirable to devise the cap, which could be manufactured simultaneously with the window, so as the window will be an integral part thereof and thus it would be possible to eliminate the necessity in additional labor, required for securing the window on the antiglare ring.

It also would be desirable to devise the cap, wherein the manufacturing and assembling with the optical head does not require strict compliance with the dimension tolerances.

For a better understanding of the present invention as well of its benefits and advantages, reference will now be made to the following description of its embodiments taken in combination with the accompanying drawings.

### BRIEF DESCRIPTION OF THE SEVERAL VIEWS OF THE DRAWINGS

FIG. 1 is an isometric, partially sectioned view of an embodiment of the cap according to the present invention.

FIG. 2 is an enlarged isometric view of the cap shown in FIG. 1 and an optical head.

FIG. 3 is a longitudinal view of the optical head with the cap put thereon.

FIG. 4 is a partial cross-sectional view of a fragment of the prior art cap showing the antiglare ring and window.

FIG. 5 is a partial cross-sectional view of a fragment of the cap according to the invention showing the antiglare ring and window.

FIGS. 6, 7, and 8 are perspective views of different embodiments of the antiglare ring used in the cap of the present invention.

FIGS. 9 and 10 schematically show the direction of light beams, defining the field of view in the prior art cap and in the cap of the invention.

### DETAILED DESCRIPTION OF THE PREFERRED EMBODIMENTS

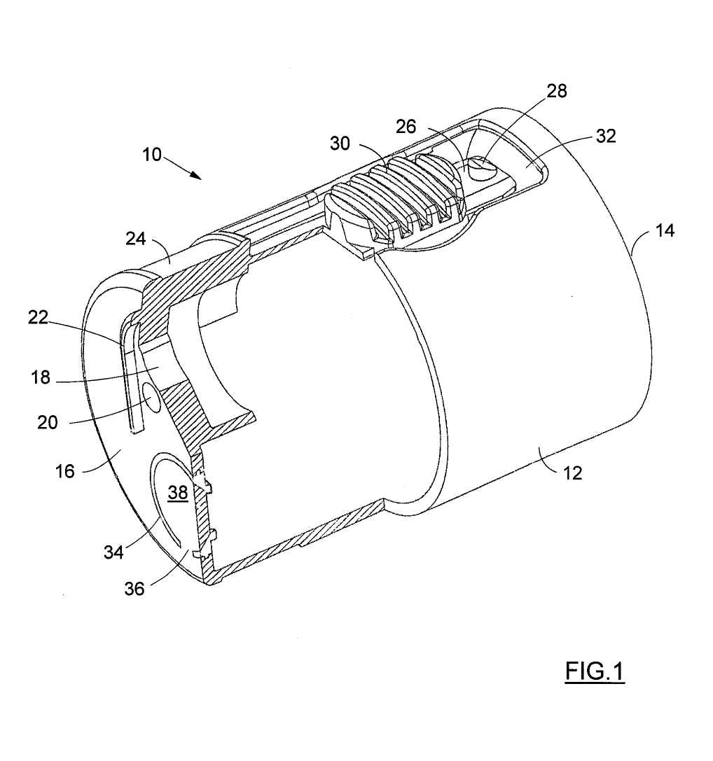

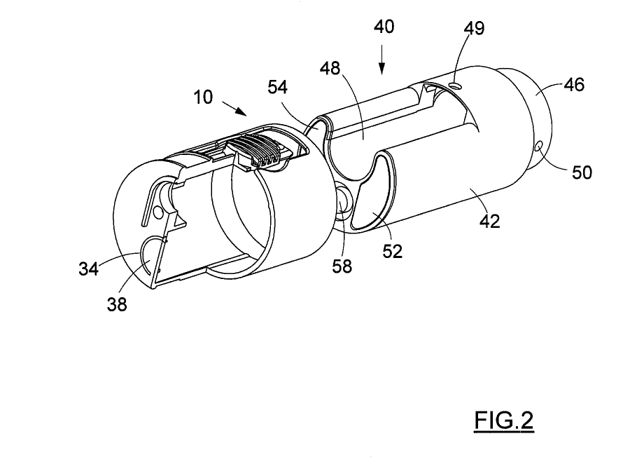

With reference to FIG. 1 and FIG. 2 an embodiment of a cap 10 of the present invention is shown. The cap is intended for use with an endoscopic apparatus, preferably colonoscopic apparatus. The components of the colonoscopic apparatus, e.g. insertion tube with working channel and navigation mechanism, operating handle, control unit, video console with monitor etc. are not shown in FIG. 1, since they are known per se. It should be appreciated that the cap accommodates therein an optical head deployed at a distal end of an insertion tube. The cap is a cheap, disposable item, which is manufactured by injection molding from a translucent plastic material, e.g. from a commercially available brand of ABS (Acrylonitrile Butadiene Styrene).

It is seen that cap 10 comprises a tubular housing 12 having a rear open end 14 and a frontal butt end closed by a frontal face 16. The interior of the housing is configured and dimensioned to accommodate therein an optical head. An opening 18 is provided within the frontal face for passing therethrough of a surgical tool when it is advanced through the insertion tube to the place of interest. This opening serves also for supplying vacuum.

It is seen also that at the frontal end an exit port 20 is provided for an irrigation channel. It is not shown in detail, but one skilled in the art would appreciate that through this port emerges a water jet, which is directed to a window made in the frontal face. The water jet is directed by a sprinkler shield (not shown) secured between guiding protrusions 22 (only left side protrusion is seen) provided on the frontal face of the cap.

Situated behind the frontal face a short depressed region 24 is provided. This region is intended for attaching thereto a distal end of a covering sleeve. It should be appreciated, however, that when the cap is used with an endoscopic apparatus, which does not employ a covering sleeve, region 24 is not necessary.

Referring to FIG. 2 it shows a cap 10 and an optical head 40 before they are connected. In practice the cap is detachably connectable to the optical head by a snap connection. As designated in FIG. 1 this connection comprises a springy tongue 26, which is cut within the housing of the cap. The tongue is fitted with a protrusion 28 and a corrugated region 30. A cutout 32 is provided around the tongue and by virtue of this provision the tongue is elastically displaceable like a lever upon pressing on the corrugated region. The optical head is provided with a hole 49, which mates with the protrusion and enables the cap to be connected to and disconnected from the optical head when the protrusion engages and disengages with the hole.

It is seen in FIG. 1, that within the lower part of the frontal face an antiglare ring 34 is embedded. In contrast to the prior art caps in the present invention this ring is not closed, but instead has a discontinuity region, i.e. a gap 36. The purpose of this discontinuity region is to permit the flow of plastic material during the injection-molding step within an area 38 encircled by the ring. By virtue of this provision the plastic material, from which the cap is manufactured, can be injected within the encircled area 38. Upon injection this area becomes an integral part of the cap's frontal face. Since the cap is made from a translucent material this area functions as a window, through which the image viewer of the optical head, i.e. projective lens of the CCD-chip or CMOS, can percept the object of interest.

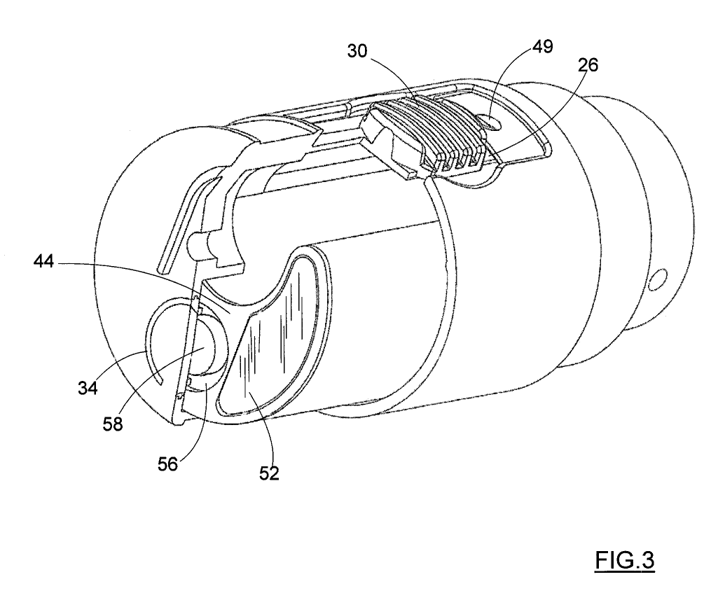

Now with reference to FIGS. 2 and 3 it is shown an optical head 40 and the cap 10. It is seen that the optical head comprises a main body portion 42 having a distal end face 44 and a proximal open end 46. That part of the main body portion, which is adjacent to the distal face 44, is provided with a U-like depression 48, while the remainder of the main body portion as well as of the proximal end 46 have a cylindrical shape. The U-like depression provides an exit for a working channel of the insertion tube (not shown).

A hole 49 is made in the cylindrical part of the main body portion. This hole mates with the protrusion 28 made on the springy tongue 26 of the cap. A bore 50 is seen, which is made on the circumference of the proximal end. This bore is intended for screws that connect the optical head with vertebrae of a navigation mechanism.

A more detailed explanation of the optical head can be found in our patent application U.S. Ser. No. 60/626,382, which is hereby incorporated herein by reference. From this explanation inter alia follows that within the main body portion are deployed two groups of illuminating means, namely white LED's. The LED's are arranged within the main body portion in such a manner that each group resides within a respective arched region provided at the left or the right side of the distal end face. Distal end face 44 is fitted with two segment-like transparent covers 52, 54 mating with the arched regions. Light generated by the LED's passes through the covers.

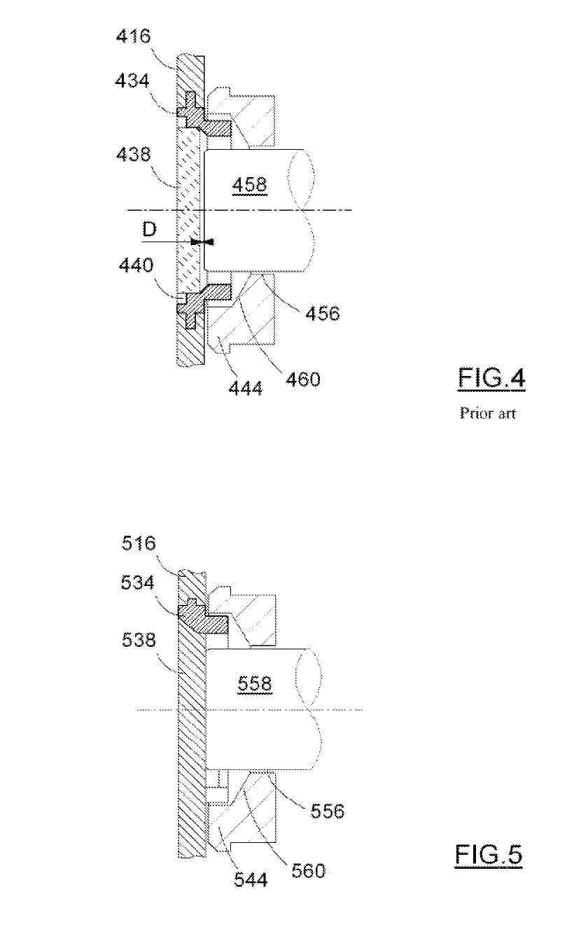

Protruding through an opening 56 in the distal end face, a projective lens 58 is seen in FIG. 3. The projective lens is protected from parasitic reflections by the antiglare ring 34 embedded within the frontal face of the cap. The antiglare ring is provided with a lower portion, which protrudes from the frontal face of the cap towards the lens. As will be apparent further from FIG. 5, an opening 56 is formed with a widening region, which is located adjacent the antiglare ring. For a better understanding of the disposition of the projective lens, of the antiglare ring and of the window, attention is directed to FIGS. 4 and 5. In these Figures three digit reference numerals are used in which the first digit designates the Figure number and the remaining digits correspond, when appropriate, to those elements, which have been already mentioned in connection with FIGS. 1, 2 and 3.

In FIG. 4 is shown a fragment of the prior art cap that employs continuous antiglare ring 434 embedded within a front face 416 of the cap. The cap is provided with a separate window 438 deployed on the ring and glued thereto. The glue is placed within an annular groove 440 surrounding the window. A projective lens 458 is seen, which protrudes along an opening 456 within a distal end face 444 of the optical head. The lens is separated from the window by a distance D. The lower portion of the antiglare ring accommodates within a widening region 460 of the opening.

In FIG. 5 is seen a fragment of the cap in accordance with the present invention. This cap employs discontinuous antiglare ring 534 embedded within a front face 516 of the cap. The front face has a region 538, which is integral therewith and which is confined by the antiglare ring. This region functions as a window. A projective lens 558 is seen, which protrudes along an opening 556 within a distal end face 544 of the optical head. The lower portion of the antiglare ring protrudes within a widening region 560 of the opening. It is seen that the projective lens is flush with the window region 538.

In accordance with the invention it has been unexpectedly revealed that despite the antiglare ring being discontinuous, it nevertheless reliably eliminates parasitic reflections and permits obtaining a coherent and sharp picture.

Now attention is called to FIGS. 6-8, which depict various embodiments of the antiglare ring provided with the discontinuity region. The ring can be manufactured from metallic material, e.g. aluminum, to provide strength, which might be required for withstanding the pressure arising when the ring is being embedded during the injection molding process.

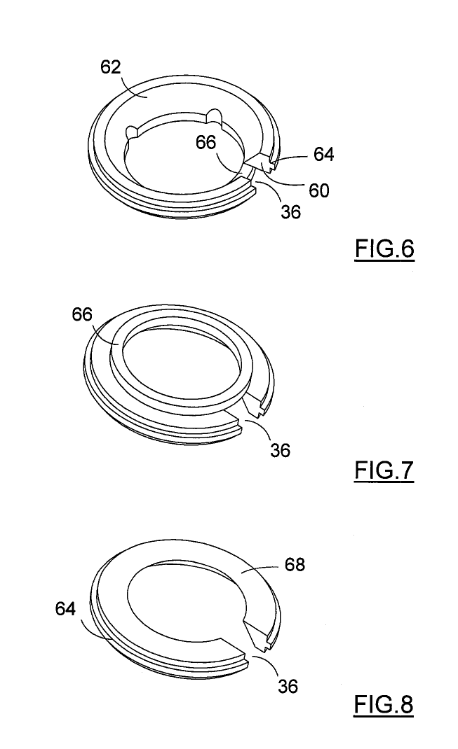

According to the embodiment shown in FIGS. 6 and 7, the antiglare ring is provided with an upper portion 60, having a conical surface 62 and a lateral ledge 64. The purpose of the ledge is to ensure steady positioning of the ring when it is embedded within the frontal face of the cap. The antiglare ring has also a lower portion 66, which protrudes from the upper portion towards the widening region of the opening in the optical head. Provided in the upper portion of the ring there is a gap 36, which renders the ring discontinuous, while the lower portion of the ring remains continuous. One should appreciate that the discontinuity region could be provided not only by means of the gap but it could be a slot, a hole drilled in the ring, etc. The discontinuity region should be sufficiently large to admit the plastic material within the area confined by the ring and to permit simultaneous injection molding of the cap and the window. To improve the injection efficiency more discontinuity regions can be provided unless they do not deteriorate the antiglare effect.

In the embodiment shown in FIG. 8 most of the ring features remain, however, the lower protruding portion is omitted and there is provided a flat lower face 68. When the cap is provided with the antiglare ring according to this embodiment the lower face of the window is flush with the projective lens.

It can be readily appreciated that, since the window can be shaped simultaneously with the cap, there is no need in attaching the window to the cap and accordingly the manufacturing process becomes less complicated, more convenient and less expensive since it requires less labor.

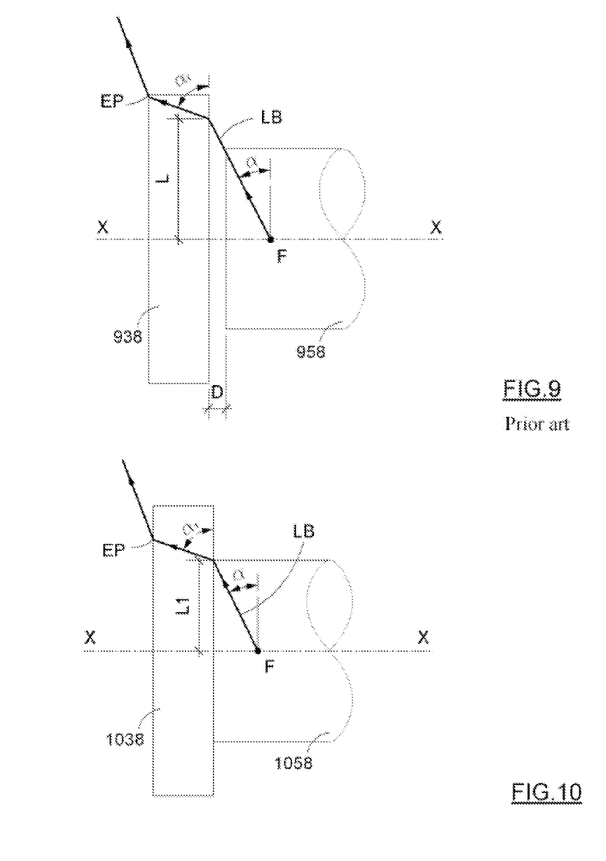

Now with reference to FIGS. 9 and 10 still further benefits of the present invention will be explained. These Figures schematically depict the field of view of the projective lens, which either is remote from the window or when it is flush with the window (as it is with the cap of the present invention). The situation depicted in FIG. 9 refers to the prior art cap in which is employed a continuous antiglare ring and a separate dedicated window. The situation depicted in FIG. 10 refers to the cap of the present invention, in which the antiglare ring is discontinuous and the window is integral with the cap's frontal face.

In FIG. 9 is schematically shown a projective lens 958 having a longitudinal axis X-X. The lens is separated from the window 938 by a distance D, which is required for preventing accidental contact between the lens and the window. A light beam LB originates from a focal point F at the lens and passes through the lens at an angle α towards the lens edge and further to the lower face of the window. The light beam approaches the lower face of the window at a distance L from the longitudinal axis X-X. Here the light beam refracts and propagates through the lens at an angle α₁, which is different from the angle α. One can see that an exit point EP, where the refracted beam exits the window, is very close to the antiglare ring. Due to this reason it is required to comply with strict dimensional tolerances on distance D and on dimensions of the window, otherwise the light beam at the exit would catch irrelevant objects in the vicinity of the ring and these objects would enter in the field of view.

In FIG. 10 is seen a projective lens 1058 having longitudinal axis X-X and window 1038. For the sake of comparison the lens and the window depicted in FIG. 10 have the same configuration and dimensions as those shown in FIG. 9.

Since the window is integral with the cap there is no danger that the window would be pushed outside. Accordingly the lens can be positioned flush with the lower surface of the window as seen in FIG. 10. Light beam LB originating from the focal point F and directed to the lens edge would approach the lower face of the window at a distance L1. Since there is no distance between the lens and the window, the distance L1 would be shorter than the distance L. Due to this reason the refracted light beam would exit from the window at an exit point EP, which is remote from the antiglare ring and accordingly less strict manufacturing tolerances need be met, since there is less probability that irrelevant objects would enter the lens' field of view.

It should be appreciated that the present invention is not limited to the above-described embodiments and that changes and one ordinarily skilled in the art can make modifications without deviation from the scope of the invention, as will be defined in the appended claims.

Below are some examples of alternative implementation of some aspects of the invention.

It is not compulsory that the antiglare ring be provided with the lower protruding portion and that the distal end face of the optical head be provided with a widening region to accommodate this protruding portion. Instead of this, one can contemplate an arrangement, in which is employed a resilient sealing ring placed between the antiglare ring and the distal end face of the optical head.

It should also be appreciated that the features disclosed in the foregoing description, and/or in the following claims, and/or in the accompanying drawings may, both separately and in any combination thereof, be material for realizing the present invention in diverse forms thereof.

We claim:

1. A cap, which is detachably connectable to an optical head of an endoscope, said cap comprising a tubular housing having a rear open end and a frontal end closed by a frontal face and an antiglare ring for preventing a viewer of the optical head from viewing parasitic reflections, wherein said antiglare ring is secured at the frontal face and said optical head can view an object of interest through a portion of the frontal face which is confined by the antiglare ring, wherein the improvement comprises said antiglare ring being provided with at least one discontinuity region.

2. The cap as defined in claim 1, wherein said cap is made of a plastic material and said antiglare ring is embedded within said plastic material, wherein said frontal face of the cap being flush with the antiglare ring.

3. The cap as defined in claim 1, wherein said at least one discontinuity region comprises a slot made in the antiglare ring.

4. The cap as defined in claim 1, wherein said antiglare ring is made of a non-transparent material.

5. The cap as defined in claim 4, wherein said antiglare ring is made of metallic material.

6. The cap as defined in claim 1, wherein said cap is detachably connectable to the optical head.

7. The cap as defined in claim 6, wherein said cap is connected to the optical head by a snap connection.

8. The cap as defined in claim 7, wherein said snap connection comprises a springy tongue, which is cut in the housing, said tongue being provided with a protrusion mating with a hole made in the optical head.

9. The cap as defined in claim 2, wherein said cap is manufactured by injection molding and said at least one discontinuity region is configured and dimensioned to permit flow of the plastic material during the manufacturing within the portion of the frontal face, which is delimited by the antiglare ring.

10. The cap as defined in claim 1, wherein said antiglare ring comprises an upper portion and a lower portion facing towards a projective lens of the optical head.

11. The cap as defined in claim 10, wherein said lower portion is flat.

12. The cap as defined in claim 10, wherein said lower portion protrudes within an opening made in a distal end of the optical head.

13. The cap as defined in claim 10, wherein said upper portion being provided with a conical configuration and with a lateral ledge.

14. The cap as defined in claim 5, wherein said antiglare ring is made from aluminum.

15. The cap as defined in claim 2, wherein said at least one discontinuity region includes plastic material within the portion of the frontal face, which is delimited by the antiglare ring.
